(19) United States
(12) Patent Application Publication
(10) Pub. No.: US 2018/0133679 A1
(43) Pub. Date: May 17, 2018

(54) METHODS OF MANUFACTURING SEMICONDUCTOR ARRAYS

(71) Applicant: Alastair J. MALCOLM, Scottsdale, AZ (US)

(72) Inventor: Alastair J. MALCOLM, Scottsdale, AZ (US)

(21) Appl. No.: 15/566,592

(22) PCT Filed: Apr. 15, 2016
(86) PCT No.: PCT/US2016/027939

§ 371 Date: Oct. 13, 2017

Related U.S. Application Data
(60) Provisional application No. 62/147,799, filed on Apr. 15, 2015.
(60) Provisional application No. 62/253,987, filed on Nov. 11, 2015.

(51) Int. Cl. B01J 19/00; B81C 1/00; C12Q 1/686

(52) U.S. Cl. CPC B01J 19/0046; B81C 1/00206; B81C 1/00896; B01J 2219/00722; B81B 2201/0214; B01J 2219/00632; C12Q 1/686

(57) ABSTRACT

A method of manufacturing semiconductor arrays is provided. A method of manufacturing semiconductor arrays may comprise applying a functionalization layer to a semiconductor wafer surface, depositing probes on the functionalized semiconductor wafer surface, and processing the printed semiconductor wafer into individual semiconductor arrays. The wafer processing steps and array finishing steps may be performed following functionalization and probe deposition in a manner that preserves the integrity of the probes.

### FIELD

The present disclosure relates to methods of manufacturing semiconductor arrays, and more specifically to methods of manufacturing semiconductor arrays with functionalization and probe deposition performed at a wafer level with subsequent wafer processing and semiconductor array finishing.

### BACKGROUND

Semiconductor arrays comprise a plurality of probes, with each probe comprising a plurality of probe elements, such as nucleic acid or protein probe elements, arranged in a predetermined pattern and physically or chemically attached to a functionalization layer on the top of a semiconductor.

Most array probes are sensitive to environmental conditions such as exposure to water, chemicals, heat, and fumes. An array may lose functionality if probes are exposed to any of a variety various environmental conditions incompatible with downstream array performance.

Semiconductor array manufacturing methods generally involve performing various final semiconductor wafer processing steps such as wafer thinning, dicing into individual die, die attachment to a circuit board, wire bonding, etc. These processes frequently involve harsh physical and chemical conditions. For this reason, wafer processing steps are generally performed prior to application of a functionalization layer and/or probes to avoid exposing the functionalization layers and/or probe elements to contamination and environmental conditions likely to deleteriously affect probe functionality.

To maintain probe functionality and semiconductor array performance, each array is individually handled and processed through functionalization, probe deposition, and additional treatments that may be required. Because of this, industrial-scale manufacturing of semiconductor arrays is inefficient and expensive. The present disclosure provides methods of manufacturing semiconductor arrays with functionalization and probe attachment performed at the semiconductor wafer level, without compromising the integrity of the probes in the semiconductor wafer processing steps.

### SUMMARY

In various aspects and embodiments of the present disclosure, a method for manufacturing a semiconductor array is provided. A method for manufacturing a semiconductor array may comprise applying a functionalization layer to a first surface of a semiconductor wafer to produce a functionalized wafer comprising a functionalized wafer surface. A method for manufacturing a semiconductor array may further comprise depositing a plurality of biological probes on to the functionalized wafer surface to produce a printed wafer comprising a printed functionalized surface. In various embodiments, a functionalization layer and a biological probe may be co-deposited in the same manufacturing step. An area of the array functionalization layer may be removed from the printed functionalized wafer surface at a plurality of locations of the functionalized wafer surface to expose the semiconductor wafer material at each of the plurality of locations. The printed functionalized wafer may be processed into a plurality of individual semiconductor arrays following functionalization and/or depositing a plurality of biological probes.

In accordance with an aspect of the present disclosure, a method of manufacturing a semiconductor array comprises applying an array functionalization layer to a first surface of a semiconductor wafer to produce a functionalized wafer comprising a functionalized wafer surface, wherein the semiconductor wafer comprises a semiconductor wafer material; depositing a plurality of probes on to the functionalized wafer surface to produce a printed wafer comprising a printed functionalized surface; removing an area of the array functionalization layer from the printed functionalized surface at a plurality of locations of the functionalized wafer surface to expose the semiconductor wafer material at each of the plurality of locations; and processing the printed wafer into a plurality of individual semiconductor arrays.

In accordance with an aspect of the present disclosure, a method of manufacturing a semiconductor array comprises applying an array functionalization layer to a wafer surface of a semiconductor wafer, wherein the semiconductor wafer comprises a semiconductor wafer material; depositing a plurality of probes on to the wafer surface, wherein the applying and the depositing steps are performed simultaneously to produce a printed wafer comprising a printed functionalized surface; and processing the printed wafer into a plurality of individual semiconductor arrays.

In accordance with an aspect of the present disclosure, processing can comprise cutting the printed wafer using a cutting technique that substantially prevents contact by the printed functionalized surface with one of liquid coolants and cutting debris. In various embodiments, processing can comprise stealth dicing. In various embodiments, cutting does not comprise one of blade dicing, laser full cut dicing, laser ablation, microjet dicing, and mechanical scribing and breaking.

In accordance with an aspect of the present disclosure, each of the plurality of probes remains substantially intact following processing.

In various embodiments, a method can comprise an array finishing step comprising one of die attachment to a substrate, attachment of an electrical connector, and application of an electrical connector protection film. The array finishing step may be performed at a finishing temperature, wherein the finishing temperature is selected based on a composition of the plurality of probes. In various embodiments, each of the plurality of probes can comprise a nucleic acid, and the array finishing step is performed at a finishing temperature of about 65° C. or less. In various embodiments, one of the plurality of probes can comprise a polypeptide, and the array finishing step is performed at a finishing temperature of about 35° C. or less.

In accordance with an aspect of the present disclosure, a method can comprise attachment of a protective chamber to a wafer prior to processing or finishing. In various embodiments, a method can comprise attachment of a protective chamber to a printed functionalized surface prior to one of a dicing, die attachment, electrical connector attachment, and electrical connector film application step. In various embodiments, a method can comprise application of a device comprising a plurality of protective chambers to a printed functionalized surface. A protective chamber can remain attached to a printed functionalized surface following an array finishing step to produce a reaction chamber.

In various embodiments, a semiconductor wafer can comprise one of an unpatterned wafer, a CMOS integrated circuit, an ISFET, a MEMS sensor, a SAW sensor, and a photodiode.

In various embodiments, a functionalization layer can comprise one of a silane film, a polymer film, or a nitrocellulose film.

In various embodiments, functionalization layer and one of a plurality of probes is deposited onto an ISFET.

In accordance with an aspect of the present disclosure, a system comprises a semiconductor wafer; a functionalization layer; and a plurality of arrays arranged on the surface of the semiconductor wafer. In various embodiments, a system can further comprise a protective chamber attached to the semiconductor wafer. In various embodiments, a system can further comprise a protective chamber device attached to the semiconductor wafer, wherein the protective chamber device comprises a plurality of protective chambers. In various embodiments, a semiconductor wafer can comprise a plurality of dies, and each die can comprise one of a CMOS integrated circuit, an ISFET, a MEMS sensor, a SAW sensor, and a photodiode. In various embodiments, each of the plurality of dies in a semiconductor wafer can be co-located with one of the plurality of arrays arranged on the surface of the semiconductor wafer.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

FIG. 1 illustrates a semiconductor array and printed semiconductor wafer in accordance with various embodiments;

FIG. 2 illustrates a semiconductor array in accordance with various embodiments;

FIG. 3 illustrates a flow chart of a process for manufacturing a semiconductor array in accordance with various embodiments;

FIG. 4 illustrates a flow chart of a process for manufacturing a semiconductor array in accordance with various embodiments;

FIGS. 5A-5D illustrate views of a protective chamber device in accordance with various embodiments of the present disclosure; and

FIG. 6 illustrates an array and a protective chamber in accordance with various embodiments of the present disclosure.

### DETAILED DESCRIPTION

The detailed description of exemplary embodiments herein makes reference to the accompanying drawings, which show exemplary embodiments by way of illustration and their best mode. While these exemplary embodiments are described in sufficient detail to enable those skilled in the art to practice the inventions, it should be understood that other embodiments may be realized and that logical, chemical, and mechanical changes may be made without departing from the spirit and scope of the inventions. Thus, the detailed description herein is presented for purposes of illustration only and not of limitation. For example, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

As used herein, a “probe” comprises one or more probe elements immobilized on or attached to a semiconductor chip or a functionalization layer applied to a semiconductor chip. A probe element can comprise a biological molecule, such as a nucleic acid sequence, an amino acid sequence, an antibody, and the like as the probe element material. A probe element can include natural or synthetic probe element materials, as described in greater detail below, and a probe can comprise a plurality of probe elements. A probe (i.e., the group of probe elements comprising a probe) may be capable of attachment to or interaction with a corresponding target from a sample, either directly or via additional intermediate molecules.

As used herein, “array” is used interchangeably with “semiconductor array” and refers to a semiconductor array, microarray or biochip that comprises a semiconductor chip and a plurality of probes attached to the chip surface, either directly or via an intermediate functionalization layer. An array may further comprise additional features functionally attached to the array, such as a printed circuit board or other device, electrical connectors, and the like. In various embodiments, an array can be used to identify particular genomic and proteomic signatures present in a sample. An array manufactured in accordance with various embodiments may be used for disease analysis, disease diagnosis and prognosis, and decision support for a course of treatment, as well as any of a variety of basic research purposes.

As used herein, a “target” is a chemical element, compound, or biological molecule in a sample derived from a human, animal, bacterium, pathogen, virus, plant, fungus, or other source that has an affinity for and/or selectively interacts with one or more probe elements. The target material may be natural or synthetic and in an unaltered state or altered state to facilitate analysis of a specimen. A target may interact with a probe element through a chemical reaction, chemical bonding, including covalent bonds, ionic bonds, hydrogen bonding, and other forms of bonding, or any other type of probe-target interaction.

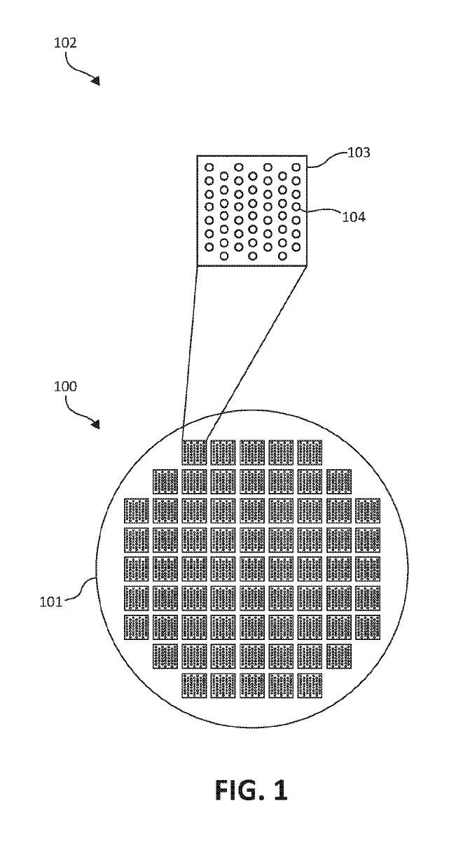

With reference now to FIG. 1, a system 100 for wafer-level manufacturing of semiconductor arrays is illustrated. In various embodiments, a system 100 can comprise a support material wafer 101. System 100 can further comprise a plurality of arrays 102 printed to the surface of wafer 101. Each array 102 can comprise support 103 and a plurality of probes 104 arranged on the surface of support 103. Each probe 104 can comprise a plurality of probe elements, as described in greater detail below, and each probe 104 may be configured to interact with a different target. In various embodiments, each array 102 in a system can comprise the same arrangement of a plurality of probes 104. Support 103 can comprise a portion of wafer 101, and can comprise various dimensions, materials and configurations. For example, wafer 101 and/or each support 103 may be porous or non-porous, and may be comprised of multiple materials, multiple layers of material, or a matrix of material, and may further comprise a two-dimensional surface or a three-dimensional porous material. When porous materials are used, the probe elements comprising a probe 104 may be located at any location on or within the support including the surfaces and interior regions of the porous material.

A wafer can comprise glass materials, colloidal materials, semiconductor materials, and plastics. In accordance with various embodiments, semiconductor wafers can comprise silicon, germanium, gallium arsenide, silicon oxide, silicon nitride, silicon carbide, silicon-germanium, and the like. A semiconductor wafer may comprise a semiconductor material that does not have a patterned integrated circuit. In various embodiments, support 103 can comprise a “chip” or “die” derived from a semiconductor wafer such as wafer 106. For example, support 103 may comprise a die with an integrated circuit, such as a complementary metal-oxide semiconductor (CMOS) integrated circuit chip, a microelectromechanical system (MEMS) chip, an ion-sensitive field-effect transistor (ISFET) chip, a surface acoustic wave sensor (SAWS) chip, a photodiode chip, and the like. A wafer used in various embodiments of the systems and methods disclosed herein can comprise a plurality of copies of an integrated circuit configuration, and the wafer can be configured to produce a plurality of dies, with each die comprising a support 103 for an array 102, and with each array printed on the wafer co-located with a die. Any type of semiconductor wafer material, whether or not subject to a microfabrication process such as doping, ion implantation, etching, deposition, photolithography, may be used in accordance with various embodiments to produce a wafer 106 comprising a plurality of arrays.

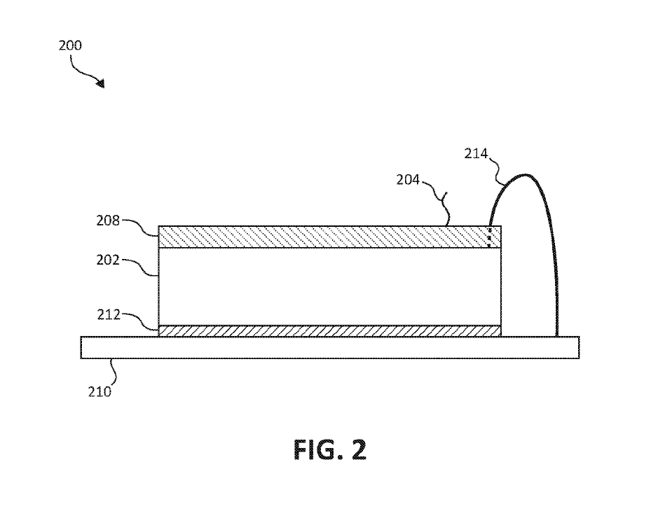

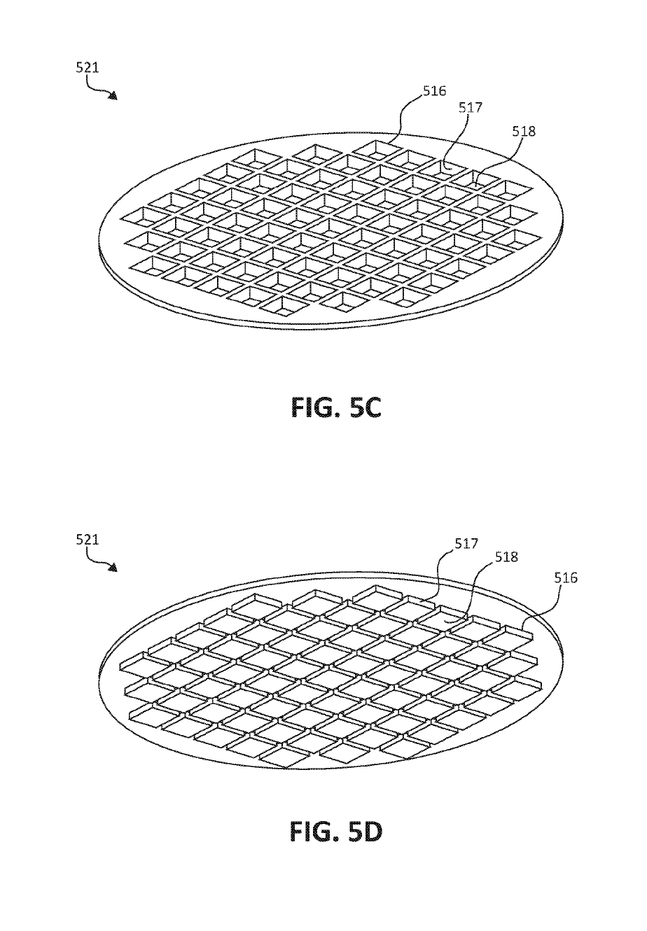

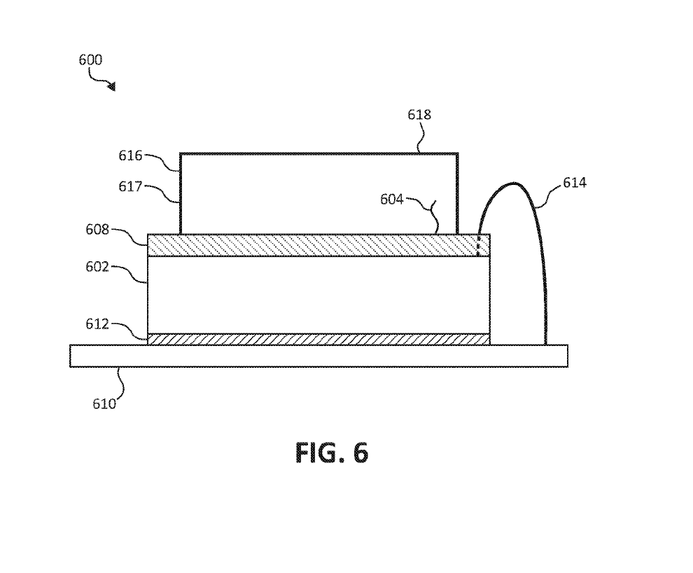

As described in greater detail below, a system for wafer-level manufacturing of semiconductor microarrays such as system 100 can further comprise a functionalization layer, such as functionalization layer 208 described below with reference to FIG. 2. In various embodiments, a system for wafer-level manufacturing of semiconductor microarrays such as system 100 can further comprise a protective chamber device or a device comprising a plurality of protective chambers, as described in greater detail below with reference to FIGS. 5A-5D and FIG. 6. A system for wafer-level manufacturing of semiconductor microarrays in accordance with the present disclosure such as system 100 comprising a plurality of arrays 102 arranged on a wafer 101 may provide various benefits such as manufacturing efficiencies when used to produce separate individual arrays 102 in accordance with various methods disclosed herein.

Probes 104 of array 102 can comprise a variety of probe element materials, such as nucleic acid sequences, for example, nucleic acid or nucleic acid analogs, including without limitation, DNA, RNA, locked nucleic acid (“LNA”) type, and peptide nucleic acid (“PNA”). The nucleic acids comprising the probe elements may be of any length, and include additional chemical groups at the ends or in the body of the nucleic acid chain. Likewise, other biological molecules and synthetic molecules may serve as probes. For example, probe elements may comprise: (i) DNA (all forms, single or double strand, natural or synthetic), (ii) RNA (all forms, single or double strand, natural or synthetic), (iii) oligonucleotides, (iv) PCR (“polymerase chain reaction”) amplicons, (v) LNA (locked nucleic acid), (vi) PNA (peptide nucleic acid), (vii) TNA (threose nucleic acid), (viii) PMO (phosphorodiamidate morpholino oligo), (ix) proteins (natural or synthetic), (x) peptides (natural or synthetic), (xi) carbohydrates, (xii) polysaccharides, (xiii) cells, (xiv) tissues, (xv) antibodies, (xvi) antigens, (xvii) protein-DNA complexes, (xviii) protein-RNA complexes, (xix) protein-protein complexes, (xx) DNA-RNA complexes, (xxi) aptamers, (xxii) dyes and dye complexes, (xxiii) stains, (xxiv) enzymes, (xxv) ubiquitin, and ubiquitinylated proteins, and (xxvi) reagents to promote probe-target reactions.

Probes 104 can be of varying dimension, shape, area, spacing and volume. Probes 104 can comprise physically separated spots produced by printing methods, for example, mechanical transfer, pin spotting, inkjet printing, acoustic printing, piezoelectric printing, electrospray printing, or any other contact or non-contact printing method.

Probes 104 may be attached to wafer 101 (and/or each support 103) by any suitable physical, chemical, or biological methods. Probes 104 may be attached to wafer 101 using probe elements that are previously prepared, and probes may be deposited in liquid, gel, or solid form. Probes 104 can be deposited onto wafer 101 via any suitable contact or non-contact printing or deposition method, such as quill pin spotting, piezoelectric spotting, electrospray spotting, ultrasonic spotting, or acoustic spotting.

In various embodiments, probe elements can be dissolved or suspended in a liquid or gel matrix (i.e., a carrier medium) for deposition onto wafer. The matrix can comprise a functionalization layer of a semiconductor microarray, described in greater detail below. Thus, in various embodiments, probes 104 may be attached to wafer 101 by co-deposition of probe elements onto wafer 101 along with the functionalization layer matrix in which the probe elements are dissolved or suspended. For example, probes 104 prepared and co-deposited in a liquid or a gel polymer functionalization layer may be specifically deposited into wells on an ISFET structure, or in close proximity to the gate regions of an ISFET transistor, and the functionalization layer matrix may be configured to provide detectable levels of signal during and following PCR cycles performed in the presence of a target, with proton release during an assay performed in the presence of the target providing the detectable signal.

In various embodiments, probes 104 may be synthesized on the support using chemical synthesis, light-stimulated synthesis, electrically-stimulated, magnetically-stimulated synthesis, enzymatic synthesis, or combinations thereof. The probes may also be conjugated with or attached to other materials, in order to provide attachment to either wafer 101, functionalization layer, a target, or intermediate molecules, discussed in more detail below.

In various embodiments and as illustrated in FIG. 2, a semiconductor array 200 may comprise a functionalization layer 208 disposed between the surface of the support 202 and the probe 204. A single probe 204 is shown in FIG. 2 as a single probe element for purposes of clarity; however, array 200 can comprise a plurality of probes, and each probe can comprise plurality of probe elements, as described herein. A functionalization layer may include one or more intermediate compounds applied to the support 202 or co-deposited with a probe 204 to achieve the desired probe attachment characteristics. Functionalization layer materials can include, but are not limited to, beads, nanofibers, nanoparticles, polymers, plastics, metals, and colloids. In various embodiments, a functionalization layer may comprise a silane film (e.g., epoxysilane, aminosilane), a polymer film, or a nitrocellulose film. Probes 204 may be deposited onto functionalization layer material 208 deposited on substrate 202, co-deposited along with functionalization layer material 208, or be synthesized in-situ with the intermediate compounds in a functionalization layer.

For example and as mentioned above, in various embodiments, probes may be co-deposited in a polymer or other functionalization layer material comprising a matrix in which the probe elements are dissolved or suspended. Without wishing to be bound by theory, co-deposition of functionalization layer material and probe element can provide an enhanced three-dimensional probe conformation in a specific region of the semiconductor device, such as a well or a gate region of an ISFET transistor used for detecting proton release and pH change. Such an enhanced three-dimensional probe conformation can produce an increased probe element density in close proximity to the sensing element of the semiconductor device, as compared to sequential deposition of functionalization layer followed by probe. For various types of functionalization layers, such as hydrogel polymers comprising a porous solid three-dimensional network with a fluid medium extender, the increased probe element density of the co-deposited probe format can produce increased signal at the sensing element due to the capacity of the hydrogel polymer for ion and/or small molecule diffusion throughout the hydrogel during a probe-target assay. Moreover, regardless of whether a sequential deposition or a co-deposition strategy is used, probe elements may further be modified to promote various aspects of probe attachment, co-location of different probe elements, or enhanced kinetics or specificity of probe-target assays.

A semiconductor array 200 may also comprise various features related to the electronic functions of the semiconductor array, such as a circuit board 210 or other support, a die attach adhesive 212 attaching support 202 of an array to circuit board 210, and one or more electrical connections 214 such as a wire bond functionally connecting the array to circuit board 210. In various embodiments and as described in greater detail below, for a semiconductor array such as array 200 comprising a functionalization layer 208, electrical connection 214 may extend through the functionalization layer 208 and make electrical contact with support 202, such as in an area where functionalization layer 208 has been removed or modified to expose the surface of support 202.

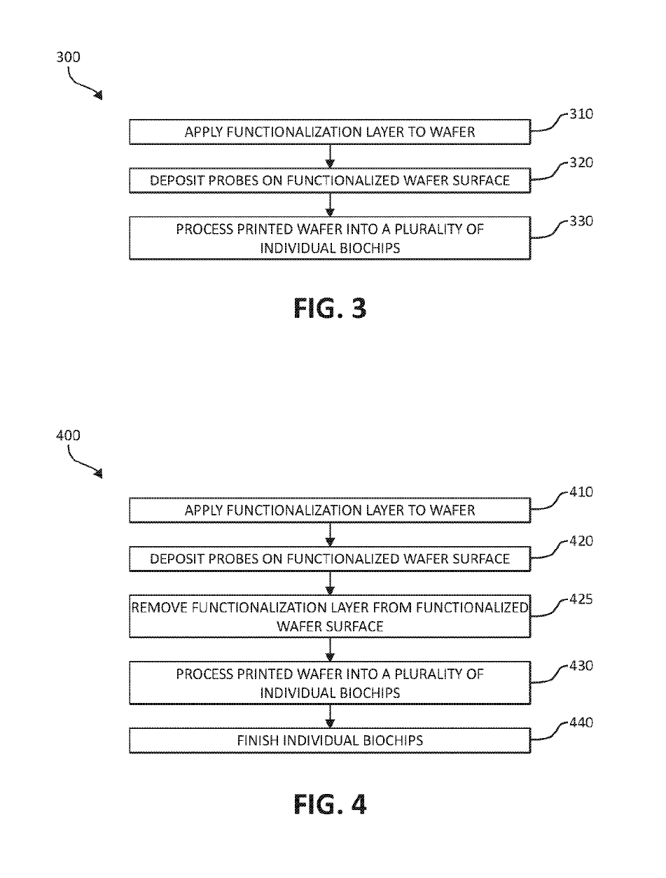

Referring now to FIG. 3, a process 300 for manufacturing a semiconductor array is illustrated. In various embodiments, a process 300 for manufacturing a semiconductor array can comprise applying a functionalization layer to a semiconductor wafer (step 310), depositing a plurality of probes on a functionalized wafer surface (step 320), and processing the printed wafer into a plurality of individual arrays (step 330).

In various embodiments, process 300 for manufacturing a semiconductor array may be initiated by applying a functionalization layer to a semiconductor wafer in step 310. As described above, a semiconductor wafer can comprise any suitable semiconductor material. A semiconductor wafer may further comprise die with patterned integrated circuits, or a semiconductor wafer may be an unpatterned semiconductor wafer material (i.e., a wafer that does not comprise microcircuits). In accordance with various embodiments, a semiconductor wafer may be subject to various preparation steps such as wafer thinning, application of a protective coating to an active circuit side (in the case of patterned microcircuit wafers), and/or oxygen plasma cleaning of the wafer prior to application of the functionalization layer. A semiconductor wafer may be coated with a functionalization layer in step 310 to assist probe deposition and adhesion to a surface of the semiconductor wafer, such as by applying an array functionalization layer to a first surface of a semiconductor wafer to produce a semiconductor wafer comprising a functionalized wafer surface. A functionalization layer may comprise one or more compounds applied to a wafer in any suitable manner, such as by silanization (including, for example, aminosilanization, epoxysilanization and the like) using any of a variety of methods that will be known to a person of skill in the art. Likewise, other functionalization layers may be applied, such as by application of polymer films, nitrocellulose films, nanoparticle coatings, and the like. Any material suitable for use as a functionalization layer to assist deposition, adhesion, and/or performance of a probe molecule on a semiconductor wafer surface, applied in any manner, is within the scope of the present disclosure.

Following application of a functionalization layer to a semiconductor wafer, process 300 for manufacturing a semiconductor array may comprise depositing probes on the functionalized wafer surface in step 320. In various embodiments, step 320 may comprise depositing a plurality of biological probes onto the functionalized wafer surface to produce a printed wafer comprising a printed functionalized surface. In various embodiments, process 300 can comprise simultaneous performance of steps 310 and 320, with co-deposition of a functionalization layer and a probe onto a wafer surface in a single step, such as co-deposition of a probe comprising a nucleic acid in a gel polymer matrix (i.e., the functionalization layer). Probes may comprise any suitable probe element material and may be applied using any suitable printing or probe deposition technique, such as those described above with reference to FIG. 1.

In various embodiments, following deposition of probes in step 320, a stabilization or activation treatment may be performed. For example, a stabilization or activation treatment can include application of a thermal, chemical, or UV illumination treatment to a co-deposited functionalization layer and probe to enable availability of the embedded probes elements to the target materials used in the assay.

Process 300 for manufacturing a semiconductor array may further comprise processing a printed wafer into a plurality of individual arrays in step 330. Processing a printed wafer may comprise dicing the wafer into individual die or arrays following functionalization and/or deposition of probes on to a wafer surface. In various embodiments and as described in greater detail below, processing a printed wafer may be performed using a dicing or cutting technique that substantially prevents formation of cutting debris, eliminates the need for a liquid coolant, maintains a relatively mild temperature range, or otherwise prevents other physical and environmental conditions, agents and/or byproducts of a cutting process from contacting the printed functionalized surface of the printed wafer.

For example, in various embodiments, a stealth dicing method may be used for processing a printed wafer into individual arrays in step 330. A stealth dicing method may comprise use of a laser with a wavelength suitable to penetrate a semiconductor wafer material, such as an infrared or near-infrared laser. The laser beam may be focused in the interior of the semiconductor wafer substrate, below the printed functionalized surface. The focused beam produces a peak power density at a focal point within the wafer thickness, forming a modified layer in the bulk of the wafer substrate without disrupting or affecting the wafer surfaces. The modified layer serves as the starting point for a crack that develops vertically in the interior of the wafer and extends upwardly and downwardly toward the front and rear surfaces of the wafer. Since the stealth dicing approach cuts the wafer from the inside, no surface debris is produced which could contaminate the functionalized surface of the wafer or the probes disposed thereon. Additionally, processing parameters may be established for stealth dicing that enable the temperature of the array to be maintained below about 40° C., thus protecting the probes from thermal damage. Stealth dicing may be performed without any requirement for liquid coolants or washes to remove debris and contamination, so no damage to the probes or functionalization layer occurs as a result of exposure to liquids. Stealth dicing, and any similar technique for processing a semiconductor wafer now known to or hereinafter devised by a person of skill in the art, may be included within the scope of the present disclosure.

In various embodiments, step 330 does not comprise one of blade dicing, laser full cut dicing, laser ablation, microjet dicing, and mechanical scribing and breaking.

In accordance with various embodiments, probes deposited on the printed wafer remain substantially intact following processing step 330. For example and as used herein, a probe may be considered “substantially intact” if it remains in a form suitable for performing downstream array experiments with an expected response and/or specificity level. In various embodiments, each individual array may be printed with one or more quality control probes. Following processing step 330, one or more individual arrays may be subjected to a quality control analysis step to assess whether printed probes remain substantially intact. In various embodiments, a quality control analysis step may determine that the individual arrays pass a quality control inspection and that printed probes remain substantially intact if, for example, at least about 80%, or at least about, 90%, or at least about 95%, or at least about 99% of quality control probes report with an anticipated level of response and/or specificity in a quality control analysis step. In various embodiments, a control probe may comprise a probe designed to be more sensitive to wafer processing and/or array finishing conditions than the experimental or test probes on an array.

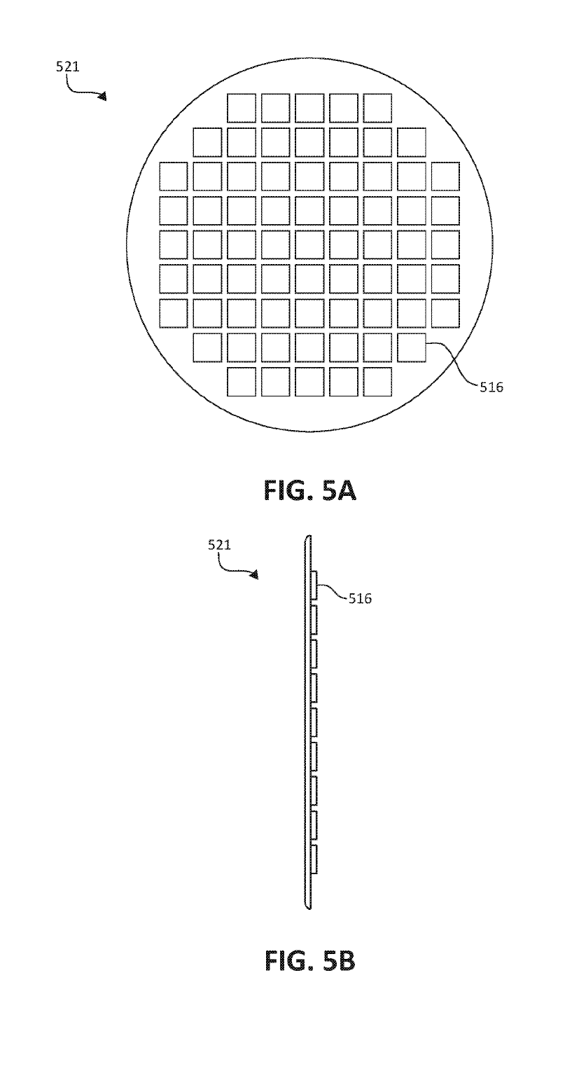

Referring now to FIG. 4, a process 400 for manufacturing a semiconductor array is illustrated. Similar to process 300 described above with reference to FIG. 3, process 400 can comprise applying a functionalization layer to a semiconductor wafer (step 410), depositing a plurality of probes on a functionalized wafer surface (step 420), and processing the printed wafer into a plurality of individual arrays (step 430). In accordance with various embodiments, process 400 may further comprise removing regions of functionalization layer from the functionalized wafer surface (step 425) and finishing individual arrays (step 440).

In various embodiments, process 400 for manufacturing a semiconductor array can comprise applying a functionalization layer to a semiconductor wafer (step 410) and depositing a plurality of probes on a functionalized wafer surface (step 420) performed as illustrated and described above with respect to steps 310 and 320 of process 300 (FIG. 3). As described above with respect to FIG. 3, in various embodiments, steps 410 and 420 may be performed simultaneously, with co-deposition of probes and functionalization layer.

Process 400 may further comprise removing functionalization layer from a functionalized wafer surface in step 425. In various embodiments, removing functionalization layer may comprise removing an area or region of functionalization layer, such as a predefined area at a discrete location (or address) of a functionalized printed surface of a wafer or an individual die located on a wafer. Removal of the functionalization layer may be performed at a plurality of discrete locations for a wafer or an individual die. Removing an area of the functionalization layer from a printed functionalized wafer surface at a location may be performed to expose the semiconductor wafer material at the respective location for attachment of an electrical connector to the semiconductor wafer material at that location. In various embodiments, removing an area of the functionalization layer may also comprise modifying (but not removing) the chemical composition or physical structure of the functionalization layer at a location. Removal of an area of the functionalization layer to expose the semiconductor wafer may be performed, for example, by laser ablation, spotting a solvent or other chemical treatment using a contact or non-contact printing method, or by any other method that may be suitable to precisely remove or alter the functionalization layer at a defined location. Removal of the functionalization layer may be performed at discrete locations near or adjacent to a probe feature with the probe feature remaining substantially intact. Any suitable method that may be used to remove or alter the functionalization layer and facilitate making an electrical connection to the semiconductor substrate is within the scope of the present disclosure.

Process 400 for manufacturing a semiconductor array may further comprise processing a printed wafer into a plurality of individual arrays in step 430. Processing a printed wafer may comprise dicing the wafer into individual die or arrays following functionalization and/or deposition of probes on to a wafer surface. Step 430 may be performed as illustrated and described above with respect to step 330 of process 300 (FIG. 3).

In various embodiments, process 400 may further comprise a step of finishing individual arrays (step 440). Finishing individual arrays 440 may comprise various steps, such die attachment to a substrate, attachment of an electrical connector to the array, application of an electrical connector protection film, and the like. In accordance with various embodiments, one or more procedures performed to finish an individual array in step 440 are performed at temperatures and/or under environmental conditions that do not substantially deleteriously affect the performance of the printed probes in downstream array experiments, such that printed probes remain substantially intact following one or more finishing steps.

In various embodiments, one or more finishing steps are performed at a finishing temperature. A finishing temperature may be controlled to remain below a certain temperature that may be incompatible with probes printed on an array. In various embodiments, a finishing temperature may be selected based on the composition of the probe printed on the array. For example, for arrays printed with nucleic acid probes, a finishing temperature of about 90° C. or less, or about 75° C. or less, or about 70° C. or less, or about 65° C. or less, or about 60° C. or less may be used. In various embodiments, the array finishing step or steps may be performed at a finishing temperature in a temperature range of about 45° C. to about 90° C., or a temperature range of about 55° C. to about 80° C., or a temperature range of about 60° C. to about 70° C., or a temperature range of about 60° C. to about 65° C. for an array comprising nucleic acid probes. For an array printed with polypeptide or protein probes, a finishing temperature of about 60° C. or less, or about 50° C. or less, or about 40° C. or less, or about 30° C. or less, or about 20° C. or less may be used. In various embodiments, the array finishing step or steps may be performed at a finishing temperature in a temperature range of about 20° C. to about 50° C., or a temperature range of about 25° C. to about 40° C., or a temperature range of about 30° C. to about 35° C. for an array comprising polypeptide or protein probes. For example, a die attach step may be performed at a finishing temperature selected from the ranges described above depending on the nature of the printed probes. Moreover, the bonding or cure time of the die attach step may be altered based on the finishing temperature selected for compatibility with the printed probes. For example, a die attach step performed using EPO-TEK 320 adhesive (Epoxy Technology, Inc., Billerica, Mass., USA) may allow a bonding period of about 12 hours at a temperature of about 35° C. for an array comprising peptide or protein probes, and a die attach step using the same adhesive may allow a bonding period of about 2 hours at a temperature of about 65° C. for a nucleic acid probe. Similarly, in various embodiments, wires from a circuit board may be attached to an array using low temperature wedge bonding or ball bonding methods comprising temperature ranges such as the temperature ranges described above during the wire attachment process. In accordance with various embodiments, any of a variety of variables involved in a finishing step, such as a material used for a finishing step, the temperature at which the finishing step is performed, and the duration of the finish step, may be selected or controlled based on compatibility with a probe element material used for an array and to preserve the integrity of the probes through the finishing process.

In accordance with various embodiments, a protective chamber may be used to protect a printed array surface during a wafer processing step or an array finishing step. In various embodiments, a protective chamber device 521 can comprise a plurality of protective chambers 516 (FIGS. 5A-5D), or a protective chamber may comprise an individual protective chamber 616 (FIG. 6). The plurality of protective chambers of a device such as protective chamber device 521 may be arranged to correspond to the arrangement of a plurality of arrays on a wafer in a system such as system 100 (FIG. 1). A protective chamber device 521 or individual protective chamber 616 can be attached to a wafer or support, via direct contact with the wafer or support material or via indirect contact with a functionalization layer between the wafer or support material and the protective chamber device or individual protective chamber. A protective chamber may comprise walls (517, 617) configured to define a perimeter surrounding an array and a ceiling (518, 618) supported by the walls. A protective chamber may be configured so that it is supported by the walls on a portion of a wafer surface or die surface located a sufficient distance from the probes of an array so as not affect the integrity of the probes. Likewise, the walls of a protective chamber may be configured to position the ceiling at a sufficient distance from the array surface so that the ceiling does not contact the probes of an array. In various embodiments, a ceiling and/or walls of a protective chamber may be optically transparent to provide visibility of the interior of the chamber to a user and/or a detection device.

In various embodiments, a protective chamber device comprising a plurality of protective chambers such as device 521 (FIGS. 5A-5D) may be applied at a wafer level to protect a plurality of printed wafer die surfaces during a dicing process. One or more individual protective chambers such as protective chamber 616 (FIG. 6) may also be applied to an individual printed wafer die surface prior to dicing, or can be applied to an individual array 600 following dicing to protect the printed array surface during various finishing steps.

In various embodiments and as illustrated in FIG. 6, protective chamber 616 can be attached to the surface of array 600. Array 600 can comprise functionalization layer 608 disposed on the surface of support 602, with protective chamber 616 enclosing one or more probes 604 (a single probe is illustrated) deposited on the surface of array 600. Array 600 can further comprise die attach adhesive 612 attaching support 602 to circuit board 610, and electrical connection 614 can provide a functional electrical connection between circuit board 610 and support 602, and can extend through functionalization layer 608 to make electrical contact with support 602, such as in an area where functionalization layer 608 has been removed or modified to expose the surface of support 602 in a process step such as step 425 (FIG. 4) of a method in accordance various embodiments of the present disclosure.

In various embodiments, a protective chamber may remain attached to an individual array. A protective chamber that remains attached to an individual array may comprise a first portion of a reaction chamber used to contain an assay, reaction, or other experiment performed using the array associated with and contained within the attached protective chamber, with the chip supporting the array comprising a second portion of the reaction chamber. An individual protective chamber attached to a printed wafer die surface or an individual array may be used to form a reaction chamber. Likewise, a single protective chamber device comprising a plurality of protective chambers may be used to provide a plurality of reaction chambers. The protective chamber device may be attached to a wafer to produce a plurality of individual arrays, each array having a protective chamber that remains attached to the array support following wafer dicing to produce a plurality separate arrays, each array having a reaction chamber. For example, a single device comprising a plurality of protective chambers such as protective chamber device 521 illustrated in FIGS. 5A-5D may be configured so that individual protective chambers may be separated from one another during the dicing process, such as by joining individual protective chambers with snap strips or other attachment means along separation lines that are substantially aligned with dicing cut locations and configured to permit separation of individual chambers along the separation lines during a dicing process. In various embodiments, a material joining a plurality of protective chambers into a single device may not be preconfigured with separation lines, but instead may be cut in the wafer dicing process to separate individual protective chambers and underlying arrays at the same time. In accordance with various embodiments, an adhesive may be used to attach a protective chamber to a wafer or array surface and to create a reaction chamber.

Manufacturing arrays in accordance with the methods of the present disclosure may permit realization of enhanced efficiencies such as reduction of material waste and handling time and increased throughput. For example, chemical costs associated with the application of the functionalization and stabilization layers and associated wash steps may be reduced by over four-fold as compared to performing these steps at an individual, assembled array level (i.e., an array die attached to a support such as a circuit board), since only the active area of the semiconductor array is required to be exposed to the chemical treatment and the underlying circuit board or support are not treated. Moreover, time and handling efficiency gains of over 20-fold may be realized in parallel processing of, for example, 1,000 semiconductor arrays arranged on an intact wafer, as compared to manipulating 1,000 individual arrays.

Likewise, the methods of the present disclosure may provide enhanced array manufacturing efficiency relative to deposition of probes. For example, probe deposition throughput may be increased by at least 20-fold, or at least 25-fold, or at least 30-fold by parallel processing of a plurality of arrays located on a single wafer as compared to serial printing of individual arrays. Deposition of the probes is typically the rate limiting step in a semiconductor array manufacturing process and requires sophisticated equipment that may represent a substantial capital investment for a business engaged in semiconductor array manufacturing. Processing the arrays in individual form limits the number of probe deposition tips that can be simultaneously utilized during manufacturing due to possible mechanical inaccuracies of over 100 microns in positioning chips on an underlying circuit board or support. In contrast, individual die arranged on an intact wafer are precisely positioned to tolerances of less than 1 micron, enabling rapid deposition of the probes to the desired location using multiple deposition tips concurrently. In this manner, probe deposition throughput can typically be increased by over 30-fold. Various other factors that may contribute to enhanced efficiencies realized by manufacturing semiconductor arrays in accordance with various embodiments will be apparent to a person of ordinary skill.

### EXAMPLES

### Example 1

An array is manufactured in accordance with the methods of the present disclosure using a printed circuit board substrate, a MEMS semiconductor chip, epoxysilane functionalization, amine-linked oligonucleotide biomolecule probes, and low temperature die attach, wire bond and glob top. This device can be used to perform on-chip polymerase chain reaction, followed by detection of the amplicons using the probes.

### Example 2

An array is manufactured in accordance with the methods of the present disclosure using a printed circuit board substrate, a CMOS semiconductor chip, polymer functionalization, amine-linked oligonucleotide biomolecule probes, and low temperature die attach, wire bond and glob top. This device can be used to perform on-chip hybridization of samples, with detection using the change in transistor photodiode electrical characteristics due to light emitted from the dye attached to the hybridized complex.

### Example 3

An array is manufactured in accordance with the methods of the present disclosure using a printed circuit board substrate, an ISFET semiconductor chip, epoxysilane functionalization, amine-linked oligonucleotide biomolecule probes, and low temperature die attach, wire bond and glob top. This device can be used to perform on-chip hybridization of samples, with detection of polymorphisms or gene sequences using the change in transistor electrical characteristics due to localized pH change of the solution near the transistor or transistors located in proximity to the probes. Co-deposition of functionalization layer and probes in close proximity to the ISFET gates enhances the performance of the device during PCR cycling and provides for release of higher levels of protons during assays, as compared to arrays constructed by deposition of probe to a functionalized array.

### Example 4

An array is manufactured in accordance with the methods of the present disclosure using a printed circuit board substrate, a CMOS semiconductor chip, aminosilane with additional activation chemistries to provide a functionalization layer, amine-linked oligonucleotide probes, and low temperature die attach, wire bond and glob top. This device can be used to perform on-chip hybridization of samples, with detection of genetic sequences or polymorphisms using the change in transistor electrical characteristics due to light emitted from bioluminescent probes attached to the hybridized complex in proximity to light sensing elements in the semiconductor.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the inventions. The scope of the inventions is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

1-21. (canceled)

22. A method of manufacturing a biochip, the method comprising:
depositing a functionalization layer material on a surface of a wafer to form a functionalization layer on the surface of the wafer;
depositing a plurality of probe elements onto the functionalization layer to form a printed wafer comprising a printed functionalized surface; and
processing the printed wafer into a plurality of individual biochips.

23. The method of claim 22, wherein the printed functionalized surface comprises a plurality of probes capable of attaching to, or interacting with, a corresponding target, each probe comprising at least one of the probe elements.

24. The method of claim 22, wherein the depositing of the functionalization layer material and the depositing of the plurality of probe elements are performed simultaneously by depositing a mixture comprising the functionalization layer material and the plurality of probe elements onto the surface of the wafer to form the printed functionalized surface of the printed wafer.

25. The method of claim 24, wherein the printed functionalized surface comprises a porous hydrogel polymer matrix with the plurality of probe elements dispersed therein.

26. The method of claim 22, wherein the wafer comprises one of a semiconductor material absent a patterned integrated circuit, a semiconductor wafer, a chip or die derived from a semiconductor wafer, a CMOS chip, an ISFET chip, a MEMS chip, a SAW chip, or a photodiode chip.

27. The method of claim 26, wherein the surface comprises a well or a gate region of the ISFET chip.

28. The method of claim 22, wherein the functionalization layer material is selected from the group consisting of beads, nanofibers, nanoparticles, polymers, plastics, metals, colloids, silanes, nitrocellulose, and mixtures thereof.

29. The method of claim 28, wherein the functionalization layer comprises at least one of a silane film, a polymer film, a polymer matrix, or a nitrocellulose film.

30. The method of claim 22, wherein the probe elements are selected from the group consisting of DNA, RNA, oligonucleotides, PCR amplicons, locked nucleic acid, peptide nucleic acid, threose nucleic acid, PMO, proteins, peptides, carbohydrates, polysaccharides, cells, tissues, antibodies, antigens, protein-DNA complexes, protein-RNA complexes, protein-protein complexes, DNA-RNA complexes, aptamers, dyes, dye complexes, stains, enzymes, ubiquitin, ubiquitinylated proteins, reagents that promote probe-target reactions, and mixtures thereof.

31. The method of claim 22, wherein the processing comprises cutting the printed wafer by stealth dicing.

32. The method of claim 31, wherein the stealth dicing further comprises focusing an infrared or near-infrared laser within an interior portion of the wafer below the printed functionalized surface to form a modified layer in the interior portion of the wafer, the laser having a wavelength capable of penetrating the wafer.

33. The method of claim 22, further comprising a preparation step preceding the step of depositing the functionalization layer material, the preparation step comprising at least one of thinning the wafer, applying a protective layer to a portion of the wafer, or cleaning the surface of the wafer with an oxygen plasma.

34. The method of claim 22, further comprising removing a region of the printed functionalized surface from the printed wafer to provide an exposed surface of the wafer, prior to the step of processing.

35. The method of claim 22, further comprising attaching a protective chamber device to the printed wafer prior to the step of processing, the protective chamber device comprising at least one protective chamber.

36. The method of claim 35, wherein the protective chamber device comprises a plurality of protective chambers arranged in a pattern to match a pattern of the printed functionalized surface on the printed wafer.

37. The method of claim 22, further comprising a finishing step performed on at least one individual biochip, the finishing step comprising one of dicing the biochip, die attaching the biochip to a substrate, attaching an electrical connector to the biochip, or applying an electrical connector protection film to the biochip.

38. The method of claim 37, wherein the finishing step is performed at a selected finishing temperature, and wherein the selection is based on a composition of the plurality of probes.

39. The method of claim 38, wherein the finishing step is performed at a finishing temperature of about 65° C. or less when each of the plurality of probes comprises a nucleic acid, and wherein the finishing step is performed at a finishing temperature of about 35° C. or less when each of the plurality of probes comprises a polypeptide.

40. The method of claim 37 further comprising attaching a protective chamber device to the printed functionalized surface prior to the step of finishing, the protective chamber device comprising at least one protective chamber.

41. The method of claim 40, wherein the protective chamber device remains attached to the printed functionalized surface following the step of finishing, and wherein each protective chamber provides an individual reaction chamber.
